(19) United States
(12) Patent Application Publication
(10) Pub. No.: US 2019/0231972 A1
(43) Pub. Date: Aug. 1, 2019

(54) Rigid Body Goggle

(71) Applicant: Phillip Cash, Dalton, GA (US)

(72) Inventor: Phillip Cash, Dalton, GA (US)

(21) Appl. No.: 16/378,636

(22) Filed: Apr. 9, 2019

Related U.S. Application Data
(63) Continuation-in-part of application No. 15/665,511, filed on Aug. 1, 2017.
(60) Provisional application No. 62/369,437, filed on Aug. 1, 2016.

(51) Int. Cl. A61M 5/142; A61B 5/145

(52) U.S. Cl. CPC A61M 5/14244; A61B 5/14503; A61M 2005/14264; A61M 5/14248; A61B 2562/18; A61B 5/14532

(57) ABSTRACT

A retaining device for use with various medical equipment attached to the body such as PODs, CGMs (Continuous Glucose Monitors) or other equipment is connected to the body assists in protecting the medical equipment, and hopefully prevents the medical equipment from being dislodged, or even lost during strenuous activity.

### CLAIM OF PRIORITY

This application is a continuation in part of U.S. patent application Ser. No. 15/665,511 filed Aug. 1, 2017 which claims the benefit of U.S. Provisional Application No. 62/369,437 filed Aug. 1, 2016, both of which are incorporated herein by reference in their entirety.

### FIELD OF THE INVENTION

The present invention relates to a protective sleeve that an individual could wear on their body to at least assist in retaining one of an insulin pump, POD or CGM (continuous glucose monitor) on the body of an individual such as on their arm or other location during sports or wet events, such as swimming, etc.

### BACKGROUND OF THE INVENTION

Insulin pumps have been around for many years. Connecting an insulin pump to a body has been the subject of numerous efforts over the years. More recently PODs have been introduced which is a smaller insulin pump typically with only a few days of insulin which normally provides an adhesive strip to attach the pod to the body of the user. Unfortunately, during sporting events, such as those which might involve physical contact, or strenuous sweating, or other activities as well, such as swimming or other water related activities, PODs tend to be dislodged, and for such events such as swimming in the ocean, can be lost.

At least one company has attempted to solve the problem at Pumpwear.com by providing a three inch wide spandex band for use in wearing over pods or sensors such as CGMs to attempt to keep them secure. They are normally sized by the arm diameter such as (a) eight to ten inch arms size for a small child, (b) ten to thirteen inch, (c) fourteen to seventeen inch, (d) sixteen to twenty-one inch, or (e) twenty-two to twenty-six inch. Accordingly, based on the size of the individual wearing the band, one of at least six different sizes can be provided. Furthermore, specific sizes are advertised as available upon request. All designs are three inch wide stretchy bands about the arm of an individual with no other structure which covers over the pod or monitor. The pre-determined resiliency and pre-determined diameter, relative to a portion of the body where the band attaches, are the only ways to select a fit of the band relative to a POD and/or user.

While this structure might be able to keep some PODs from becoming lost, they certainly are not normally waterproof and the POD may become dislodged internal to the band during strenuous activity and/or swimming. This would then require re-inserting the needle to deliver insulin. Furthermore, this type of construction does not lend itself to cover a multiple attachment locations, such as an arm, torso or leg of a user. Furthermore, the user would appear to be stuck with the design provided on the band. In order to have a new design, a new band would need to be provided. Additionally, the website of Pumpwear.com advises that when ordering these bands that “bands are made to be snug so that they keep pods and sensors secure, however it is your responsibility as the user to insure the pods and sensor do not come off and Pumpwear, Inc. is not responsible for sensors or pods that come lose.”

Accordingly, there is a perceived need for improved constructions in the marketplace.

### SUMMARY OF THE INVENTION

Accordingly, it is an object of a presently preferred embodiment of the present invention to provide an improved cover for retaining at least one of a continuous glucose monitor, pod and/or insulin pump against the body of a user preferably in an attached manner;

It is also an object of many embodiments of the present invention to provide an improved sleeve with a cooperating goggle structure which can provide at least a water-resistant, if not waterproof seal, against the skin of a user about an attached medical device to thereby assist in reducing the amount of water onto the device (such as one of a POD, CGM and/or insulin pump) during sports, strenuous activity and/or wet use.

It is also an object of many embodiments of the present invention to provide an improved protection device for protecting an installed pod, continuous glucose monitor and/or insulin pump on a person.

It is an object of many embodiments of the present invention to provide a cover which can retain an installed pod, glucose monitor and/or insulin pump, even if dislodged from its intended location.

Accordingly, in accordance with presently preferred embodiments of the present invention, a goggle-type structure is provide which provides at least a water-resistant seal relative to the skin of a user with a perimeter preferably about or relative to a perimeter of a medical device having a needle stuck into the skin of a user such as one of a POD, CGM or insulin pump (other devices, any deliver glucose, chemotherapy or other medication or check other blood parameters with similar devices), whereby the cover can provide at least a water-resistant, if not a waterproof, seal together with the skin to assist in not only retaining the device within the structure, preferably in a water-resistant, if not waterproof, manner, so as to prevent the device from being lost, but also assist in maintaining the device in position in an installed configuration with the needle connected to the device inserted by the person's skin, preferably internally to the cover.

The goggle can be connected to the skin in a number of ways. It is envisioned that a band connects the cover to at least one of a portion of the person whether that be an arm, a torso, or a leg of an individual, although certain other body parts could at least assist in connecting the structure to the body of a user as well.

For some embodiments a first seal layer can be applied against the skin. The cover may be adjacent thereto, such as connected or molded thereto, so as to assist in providing a water-resistant, if not waterproof, seal with the cover relative to the skin in an effective manner which being retained to the body by the band. The first seal placed against the skin may be made of a resilient material or not, possibly a latex, rubber, etc. onto which a cover can be adjacently disposed. Some embodiments may have a first layer integrally provided with the cover.

The cover may take a variety of forms whether as a hard material and/or a flexible material depending on the application. Many preferred embodiments will utilize a rigid cover.

Some embodiments may utilize belts or bands to retain the goggle to a body part. Belts and bands may take various forms.

The belts or band preferably has a span of cover separating ends of the band therebetween which could be similar or dissimilar to prior art constructions. The band could be tightened (or loosened), or not, and allow the span to cover the medical device and then retained in position. Should the medical device become dislodged, it preferably would be retained between the belts internal to the span. The hard cover, if utilized, will hopefully protect the medical device from becoming dislodged, and a waterproof seal will hopefully keep it fully functional during and after wearing the goggle.

### BRIEF DESCRIPTION OF THE DRAWINGS

The particular features and advantages of the invention as well as other objects will become apparent from the following description taken in connection with the accompanying drawings in which:

FIG. 1 is a side plan view of a presently preferred embodiment of the present invention with a medical device shown in phantom inside connected to a user;

FIG. 2 is a bottom plan view of the embodiment of FIG. 1 without the attached band;

FIG. 3 is a top perspective view of the embodiments of FIGS. 1 and 2; and

FIG. 4 is a side plan view of the embodiment of FIG. 1 installed on a from the user;

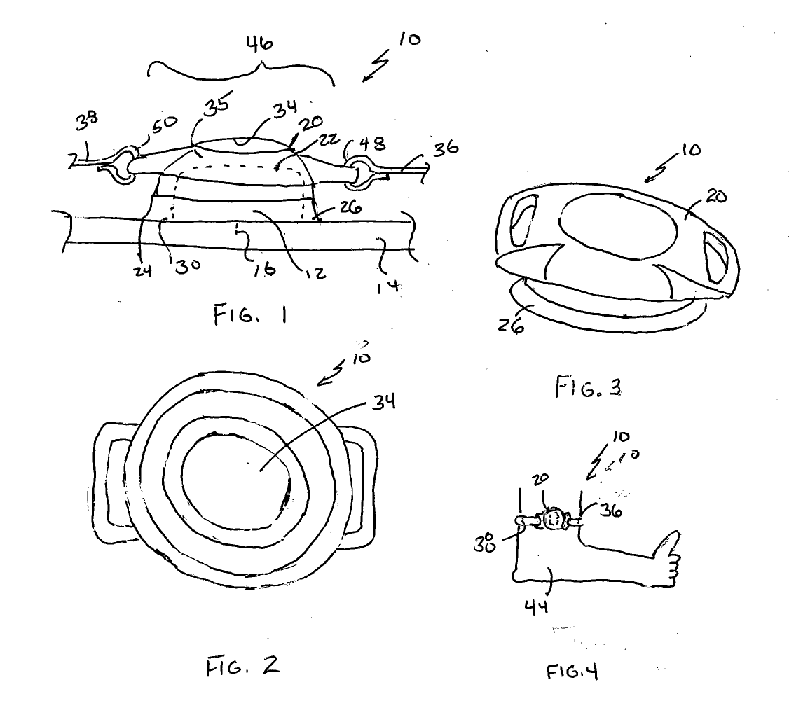

### DETAILED DESCRIPTION OF THE PREFERRED EMBODIMENT

The figures show a protective goggle illustrated as a goggle 10 having a construction calculated to at least assist in protecting a medical device 12 having a penetration through the skin 14 of a user, such as illustrated. Medical devices 12 could be insulin pumps, PODs, Continuous Glucose Monitors (CGMs), chemotherapy pumps, glucose pumps and/or other delivery and/or sensor systems which have penetrations such as a needle 16 extending into the skin 14. For this application, “connected medical devices” are defined as medical devices 12 having a continuous penetration through the skin 14 and are connected to a supply or monitoring apparatus like a pump, glucose monitor, etc. “Disconnected medical devices” could refer to feeding tubes, IV ports, and the like, which are disconnected from a source, and need to be re-attached to be functional.

Goggle 10 preferably provides a cover 20 which may take a variety of forms but some embodiments preferably extend above an outer or upper most surface 22 of the medical device 12 and extend to a perimeter 24, possibly of a foot 26, which may, or may not directly contact the skin 14, depending upon the embodiment. Cover 20 may be transparent or translucent to be able to see the medical device 12, when installed (which could be helpful to be able to read a number or other indicia, such as a reading on the CGM or other medical device 12), or not. Cover 20 may also be at least partially flexible or rigid, depending upon the embodiment.

While foot 26 may directly contact skin 14 for some embodiments, it may not for other embodiments.

Foot 26, as illustrated, may be able to contact skin 14 and assist in forming a water resistant, if not water proof, seal with the skin 14 about at least the penetration at the perimeter 24 which might be substantially circular or other shape, illustrated as needle 16, if not about a perimeter 30 of the medical device 12 to provide an air cavity about the device 12 such as in the gap 35 and internal areas of the cover 20 relative to the skin 14. Since many medical devices, such as PODs are stuck onto the skin, having the cover contact the skin 14 at a first pre-determined perimeter 32 about the perimeter 30 of the medical device 12 can keep the medical device 12 secured to the skin 14 as it is designed to do. Inner surface 34 of cover 20 may contact the upper most surface 22 of the device, or not, such as by being spaced by gap 35 above upper most surface 22 while the cover 20 may still surround the medical device 12 with the skin 14.

Unlike spandex bands of the prior art, the cover 20 may, or may not, apply downward pressure against the medical device 12, or not, potentially independently of tension provided to the bands 36,38 and/or others utilized to secure the protective sleeve 10 to a body portion of the user, such as arm 44, leg, torso or other appropriate body location. Some embodiments may maintain gap 35 above all of the uppermost surface 22 of the device 12 so that cover 20 does not contact uppermost surface 22 and may not even contact medical device 12 at all.

The cover 20 may have first and second ears 40,42 extending cantileveredly away from the cover 20. The ears 40,42 may be located below an upper and/or uppermost surface 24 of the cover 20 and even extend outwardly beyond the perimeter 24 of the foot 26 for at least some embodiments. Span 46 may space apart ends 48,50 of the bands 36,38. Bands 36,38, may be part of the same band or different bands connected together.

The cover 20 may be held in place in a variety of manners as known in the art while still assisting in providing both a span 46 about the medical device 12 to assist in protecting the medical device 12 from dislodgement of the penetration, such as needle 16 from the skin, while also potentially protecting the medical device 12 from being lost, or even exposed to too much moisture so that adhesive of the device as connected to the skin might not be compromised at a failing rate for at least some embodiments, such as if the user is swimming or engaged in certain activities.

Some bands 36,38, may take on a variety of widths. The bands 36,38 can have a variety of connections and constructions.

By having bands 36,38 being capable of providing an adjustable diameter such as about arm 44 as illustrated, such as by being at least partially resilient or elastic, but also about torso and/or legs, a variety of more options can be explored by the user than the prior art spandex bands having at most a six inch variance in the body part a particular band might provide. Thus, bands 36-38 circumnavigate a body part with the span 46 of the cover 20 when installed to locate the cover 20 over a device 12.

For at least some embodiments, any of bands 36,38 could be detachably connected to either of the ears 40,42 so as to be able to swap out bands 36,38, relative to the ears 40,42 and/or cover 20, to account for different colors, designs and/or lengths.

The covers 20 may, or may not, be manufactured to cooperate with specific medical devices 12 such as by having an inner surface 50 calculated to either contact or not contact the medical 12 at certain locations and/or be appropriately spaced therefrom. Of course, covers 20 could be at least somewhat generic to the medical device(s) they retain.

The span 46 of the cover 20 is illustrated extending beyond the perimeter 30 of the medical device 12 (and possibly beyond perimeter 24 of foot 26).

One can quickly recognize the benefits of the goggle 10 over prior art constructions in being able to protect medical devices 12 without necessarily applying pressure to the medical device 12 when tightening any of the bands 36,38, for adjustable tightness of the bands 36,38, without necessarily applying downward pressure on the device 22 against the skin 14 if even contacting the medical device 12 at all with the goggle 10 fully installed. Not only can wet activities, like swimming, waterskiing, jet skiing, surfing, or other water related activities be enjoyed while limiting contact of external water with the medical device 12, but also physical activities like sports (basketball, tennis, and/or others) could be enjoyed with the medical device 12 more likely to be retained in position with the sleeve 10 so that the user need not worry if it might be dislodged during those activities.

Various embodiments could take a variety of forms, such as whether the google 10 and/or any of the bands 36,38 could be disposable or not. They could be designed for a significant life span, or not. The cover 20 could be disposable or not. Many embodiments of the goggle 10 will be at least water resistant, if not water proof.

Numerous alterations of the structure herein disclosed will suggest themselves to those skilled in the art. However, it is to be understood that the present disclosure relates to the preferred embodiment of the invention which is for purposes of illustration only and not to be construed as a limitation of the invention. All such modifications which do not depart from the spirit of the invention are intended to be included within the scope of the appended claims.

Having thus set forth the nature of the invention, what is claimed herein is:

1. A method of using a protective device comprising the steps of:
providing a connected medical device having a penetration through the skin of a user connected to at least one of a supply, a pump and a monitor;
covering the connected medical device with a goggle,
said goggle comprising
a cover, said cover extending over an upper most surface of the connected medical device external to the user and beyond a perimeter of the connected medical device when connected, and
a foot having a predetermined perimeter, said foot forming at least a water resistant connection against the skin of the user under the cover, said foot connected to the cover; and
securing the goggle to a body part with a band, said band connected to cantileveredly extending ears of the cover whereby first and second ends of the band are spaced by a span of the cover apart.

2. The method of claim 1 wherein the connected medical device is selected from the group of insulin pumps, PODs, continuous glucose monitors, chemotherapy pumps, and glucose pumps.

3. The method of claim 1 wherein the cover is one of translucent and transparent.

4. The method of claim 1 wherein the cover is one of flexible and rigid.

5. The method of claim 1 wherein the cover is spaced by a gap from the upper most surface of the connected medical device when covering the connected medical device.

6. The method of claim 1 wherein the goggle contributes no downward force to the connected medical device when covering the connected medical device.

7. The method of claim 1 where the seal is a waterproof seal.

8. The method of claim 7 further comprising a gap between the upper most surface of the connected medical device and the cover when covering the connected medical device with the goggle, with an air cavity located in the gap about the connected medical device, and an uppermost surface of the cover extends above the ears.

9. The method of claim 1 wherein the foot of the cover against the skin forms a watertight seal.

10. The method of claim 1 wherein the body part is selected from one of an arm, torso and legs.

11. The method of claim 10 wherein the band provides an adjustable diameter about the body part.

12. A method of using a protective device comprising the steps of:
providing a connected medical device having a penetration through the skin of a user;
covering the connected medical device with a goggle,
said goggle comprising
a cover having cantilevered ears connecting to opposing ends of a band spaced apart by a span below on uppermost surface of the cover, said cover extending over an upper most surface of the connected medical device external to the user, and
a foot contacting against the skin of the user under the cover, said foot connected to the cover, so that the google forms at least a water resistant seal about the connected medical device when installed.

13. The method of claim 12 wherein the at least water resist seal is a water tight seal.

14. A method of using a protective device comprising the steps of:
providing a connected medical device having a penetration through the skin of a user;
covering the connected medical device with a goggle,
said goggle comprising
a cover, said cover extending over an upper most surface of the connected medical device external to the user spaced by a gap, said cover having ears extending cantileveredly beyond a perimeter of the medical device when installed, and
a foot forming a seal against the skin of the user under the cover, said seal connected to the cover.
